(19) United States
(12) Patent Application Publication
(10) Pub. No.: US 2025/0302514 A1
(43) Pub. Date: Oct. 2, 2025

(54) Sterile packaging for a sterile object

(71) Applicant: Karl Leibinger Asset Management GmbH & Co. KG, Mühlheim an der Donau (DE)

(72) Inventors: Jörg Henke, Mühlheim an der Donau (DE); Martin Pabst, Mühlheim an der Donau (DE)

(21) Appl. No.: 19/097,566

(22) Filed: Apr. 1, 2025

(30) Foreign Application Priority Data
Apr. 2, 2024 (DE) 102024109157.6

(51) Int. Cl. A61B 17/86; B65D 75/36; B65D 75/58; B65D 77/04

(52) U.S. Cl. CPC A61B 17/865; B65D 75/366; B65D 75/5855; B65D 77/0413

(57) ABSTRACT

Sterile packaging for a sterile object (40) includes an outer packaging (1) and at least one container (10) arranged in the outer packaging (1) that forms a sterile barrier and in which a receiving compartment (13) is formed. The container (10) includes a first tray (15) closed by a peal-off first lid (14) and features a first recess in which the receiving compartment (13) is formed. The receiving compartment (13) is adapted to receive the sterile object (40) directly or indirectly. The outer packaging (1) features the form of a prism having two triangular or rectangular faces (1a, 1b), which are opposite each other along the longitudinal direction and which form a bottom face (1a) and a top face (1b) of the prism and also having three or four rectangular lateral faces (1c) forming a lateral surface area of the prism.

### CROSS-REFERENCE TO RELATED APPLICATION

The present application is related and has right of priority to German Patent Application No. DE102024109157.6 filed on Apr. 2, 2024, which is incorporated by reference in its entirety for all purposes.

### TECHNICAL FIELD

The present invention relates generally to a sterile packaging for a sterile object, in particular for an implant.

### BACKGROUND

DE 103 05 948 A1 describes a packaging for a dental implant. The packaging features a rigid casing with an implant holder arranged inside. The casing is stable at two different levels and features an opening to access the implant holder. The implant holder features an indentation to accommodate the implant.

EP 2 896 580 A1 describes a blister packaging with an interior compartment. The blister is formed with the lateral faces of a prism, with at least one of the prism's end faces being closed or designed to be closed.

### BRIEF SUMMARY

Example aspects of the invention provide a sterile packaging for a sterile object, which is characterized by particularly easy handling when opening as well as a high packing density when stacking several similar sterile packagings.

A sterile packaging for a sterile object, such as an implant is proposed. The implant can, for example, be a screw. The sterile packaging features an outer packaging as well as at least one container arranged in the outer packaging. Thereby, the container forms a sterile barrier. A receiving compartment is formed in the container, which is designed to directly or indirectly receive the sterile object. The container features a first tray, which is closed by a first lid. The container can be opened by pulling off the first lid.

According to example aspects of the invention, the outer packaging is designed in the form of a prism which features two triangular or rectangular lateral faces that face each other in the longitudinal direction of the outer packaging. Hereby, the two lateral faces form a bottom face and a top face for the prism. The lateral surface area of the prism is formed by further rectangular lateral faces. If the bottom face and top face are formed as triangles, the lateral surface is formed by three rectangular lateral faces. If the bottom face and top face are formed as rectangles, the lateral surface is formed by four rectangular lateral faces.

The design of the outer packaging as a prism according to example aspects of the invention allows several such outer packagings to be stacked in a particularly space-saving manner. The arrangement of a container closed by a lid inside the outer packaging simplifies handling when the sterile object is removed from the sterile packaging. By opening the outer packaging, the container can be removed and opened by pulling off the lid.

The container can directly accommodate the sterile object, so that a simple sterile barrier is given. Alternatively, the container can hold a further container, thus providing a double sterile barrier.

When the bottom face and the top face are in the form of a rectangular prism, it is preferable to provide that two opposite lateral lengths of the bottom face and the top face are of different lengths, so that one of the lateral lengths is at least three times as long as the other of the lateral lengths. This type of design allows the outer packaging to be made particularly stable, while still allowing a high packing density when stacking several sterile packages.

Preferably, the bottom face and the top face feature the shape of a triangle, particularly preferable is the shape of a right-angled triangle. This design also allows for a particularly high packing density when stacking several such sterile packagings.

The outer packaging can be opened by depressing a predetermined breaking point perforation located at an end section of the outer packaging, for example with a user's finger and raising a lid.

According to a preferred example embodiment, the container is configured as an outer container that forms a first sterile barrier. The outer container can, for example, be easily removed by simply pulling from the outer packaging. The outer container can be held at a first end section for opening with a first hand, in particular with the thumb and index finger, whereby the first lid is pulled off with the other hand starting from a second end section of the outer container that is opposite to the first end section. When doing so, the first sterile barrier is opened. In this example design, the first receiving compartment contains an inner container which forms a second sterile barrier. During and after pulling off the first lid, the inner container can continue to be kept sterile with the first hand on the first end section of the outer container, so that, for example, unintentional falling out of the inner container can be avoided. In this condition, a transfer of the sterile inner container by the support staff in the operating room, which is considered “non-sterile”, to a second person, who is specifically in a sterile area, e.g. a surgeon or similar, can take place. The inner container includes a second tray with a second recess, which is closed by a peel-off second lid. A second receiving compartment is incorporated in the second recess. The second recess features an indentation that rests on a surface of the first recess in the first receiving compartment as a pivotal point for a tilting movement of the inner container relative to the outer container. By applying pressure to the inner container while holding the opened outer container at the first end section, the inner container tilts around the pivot relative to the outer container, allowing the inner container to be removed without touching the outer container. For this purpose, the inner container can be grasped at a first end section opposite the pivotal point. The tilting motion thus enables particularly safe and easy removal of the sterile inner container from the outer container. Thus, contamination of the inner container can be avoided.

The sterile object or a carrier for the sterile object can be arranged in the second receiving compartment. Removal of the carrier, respectively the sterile object, from the inner container is performed in a manner similar to the removal of the inner container from the outer container by holding the inner container at the first end section and pulling off the second lid, starting from a second end section of the inner container that is opposite the first end section. Then the sterile object or carrier can be removed easily and safely. Opening of the inner container is usually performed in a sterile environment.

In line with a preferred example embodiment, the first tray includes a peripheral edge section that surrounds the first recess and to which the removable first lid is attached.

In a similar manner, the second tray can feature a peripheral edge section that surrounds the second recess and to which the removable second lid is attached.

The preferred edge section of the first tray and the second tray forms a flat surface framing the respective recess for attaching the respective lid. Furthermore, shaping of the edge section around the recess can increase the rigidity of the first and second trays respectively.

Preferably, the sterile packaging extends in a longitudinal direction. The outer container preferably features a suitably fitting shape so that the outer container can be inserted precisely into the outer packaging. With particular preference, additional space can be provided in the outer packaging for a package insert or similar.

The first tray preferably features a first end section for retaining the outer container and a second end section opposite the first end section in longitudinal direction. The end sections can each be formed as part of the preferred edge section. The end sections are designed in a suitable manner for easy grasping of the outer container between the thumb and index finger.

The first recess is preferably formed in longitudinal direction between the first end section and the second end section. Furthermore, a less pronounced section can be formed between the second end section and the first recess to allow the second end section to be kinked. For example, the thickness of the tray material can be reduced in the less pronounced section to allow easy kinking or bending of the second end section. Removal of the inner packaging can be further simplified by bending, respectively kinking, the second end section. Furthermore, kinking can simplify removing of the first lid.

Similarly, the inner packaging can also feature a less pronounced section for kinking a corresponding second end section. On the one hand, this can make it easier to remove the second lid. On the other hand, it can make it easier to remove the sterile object or carrier from the inner packaging.

According to a preferred example embodiment, the outer packaging can be designed as a cardboard box. Preferred in particular, the outer packaging can feature a predetermined breaking point line for opening the outer packaging. The predetermined breaking point line can, for example, be designed as a perforation that runs around at least part of the circumference of the outer packaging, so that breaking the perforation creates a removable or hinged lid. To make it easier to break the perforation, a cut-out or perforated pressure point can be formed on the surface of the outer packaging in the vicinity of the predetermined breaking point line. Furthermore, the outer packaging can preferably feature a transparent window through which the contents of the outer packaging can be seen.

According to a preferred example embodiment, a carrier is arranged in the second receiving compartment, which features a shaped part with a third receiving compartment for the sterile object. For example, the molded part can be designed to exert a clamping effect on the sterile object to keep the sterile object in place. This can prevent the sterile object from accidentally falling out of the molded part. In preferred example embodiments, the sterile object may be, in particular, a bone screw or the like.

### BRIEF DESCRIPTION OF THE DRAWINGS

Example aspects of the invention are described in detail on the basis of the figures. Illustrated in:

FIG. 1 a perspective view of a prism-shaped outer packaging;

FIG. 2 a perspective view of the prism-shaped outer packaging, as well as the corresponding outer container and inner container;

FIG. 3 a lateral view of an outer container with a tilted inner container;

FIG. 4 a perspective view of an inner container with a tilted carrier retaining a sterile bone screw;

FIG. 5 a sectional view through the outer container with tilted inner container;

FIG. 6 a sectional view through the inner container with tilted carrier; as well as

FIG. 7 a perspective view of a further example embodiment of the outer packaging.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

The accompanying figures are intended to provide a further understanding of the embodiments of the invention. They illustrate embodiments and, in the context of the description, serve to explain the principles and concepts of the invention. Other embodiments and many of the advantages mentioned arise with regard to the drawings. The elements of the drawings are not necessarily drawn to scale in relation to each other.

In the figures of the drawing, the same, functionally identical and similarly acting elements, features and components are each provided with the same reference signs-unless otherwise stated.

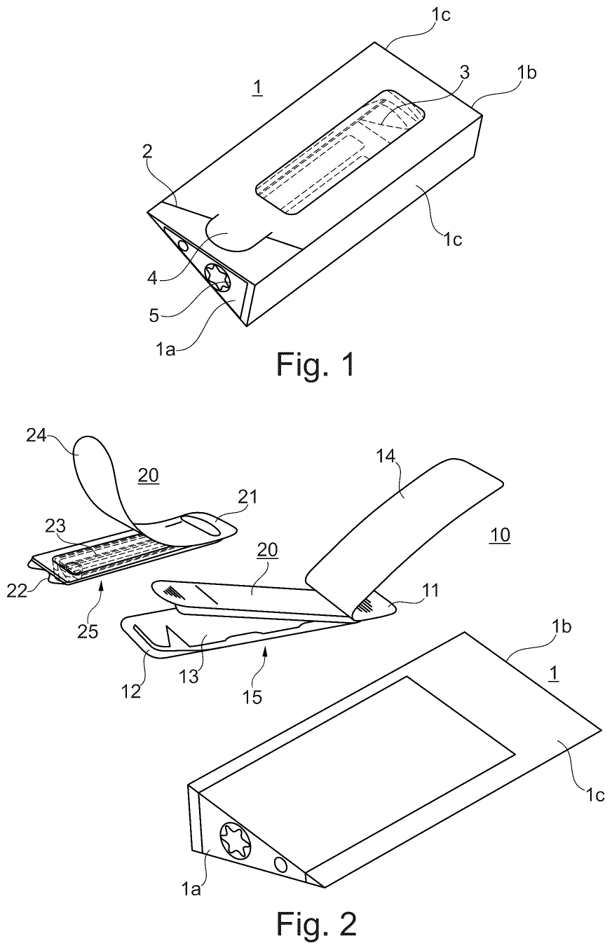

FIG. 1 shows a perspective view of an outer packaging 1 of a sterile packaging according to an example embodiment of the invention. The outer packaging 1 features a perforated predetermined breaking point line 2 that is broken by pressing at the pressure point 4 to open the lid 5. In the example shown, the predetermined breaking point line 2 extends along three faces of the outer packaging 1, so that the lid 5 remains hinged to the fourth side. Furthermore, the outer packaging 1 features a window 3 into which a transparent film is adhesive-bonded. This allows the contents of the outer packaging 1 to be viewed. The prism-shaped outer packaging 1 features a triangular cross-section. In other words, the bottom face 1a and the top face 1b of the prism have the shape of a right-angled triangle. Consequently, a large number of such sterile packagings can be stacked or packaged and stored in a particularly space-saving manner.

FIG. 2 shows a perspective view of the prism-shaped outer packaging 1 as well as a corresponding outer container 10 and inner container 20, which can be arranged inside the outer packaging 1. The outer container 10 and inner container 20 feature a cross-sectional shape corresponding to the triangular cross-section of the outer packaging 1.

The center view of FIG. 2 shows the outer container 10 with the lid 14 partially removed from the second end section 12 in the direction of the first end section 11. The inner container 20 is shown in a tilted position in which the sealed sterile inner container 20 can be removed particularly easily and without contamination from the outer container 10. By holding the outer container 10 at the first end section 11 and pressing on the first end section 21 of the inner container 20, the inner container 20 tilts around a pivotal point. An indentation 26, for example, can serve as a pivotal point, resting on a surface in the receiving compartment 13 of the outer container 10.

At the top left, FIG. 2 shows the inner container 20 with the lid 24 partially removed from the second end section 22 in the direction of the first end section 21, so that a second receiving compartment 23 can be seen. In the second receiving compartment 23, a carrier for a sterile object or the sterile object without a carrier can be arranged.

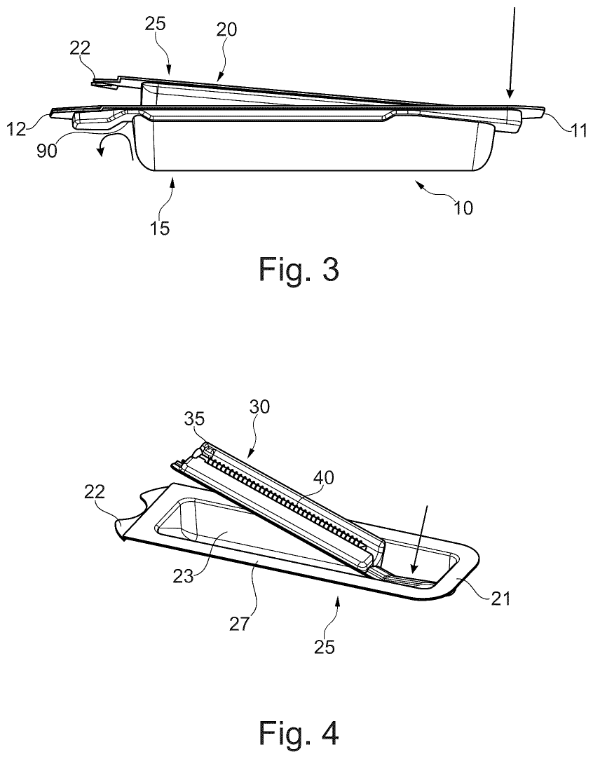

FIG. 3 shows a side view of the outer container 11 from FIG. 2 with a tilted inner container 20. A representation of the lid 14 was refrained from in FIG. 3. The arrow indicates the direction of a force for exerting pressure on the first end section 11. This force also acts on the first end section 21 of the inner container 20 and causes the inner container 20 to tilt relative to the outer container 10, so that the inner container 20 partially protrudes out of the outer container 10. Thus, the sterile inner container 20 can be easily removed from the outer container 10 by grasping the second end section 22.

The curved arrow in FIG. 3 illustrates a kinking movement of the second end section 12 at a thinned section of the first tray 15. For example, a user grasps the first end section 11 with the thumb and index finger of the right hand and the second end section 12 with the thumb and index finger of the left hand, tilting the inner container 10 outwards by applying pressure between the thumb and index finger of the right hand and bending the second end section 12 downwards. This way, a second person in a sterile area can easily grasp and remove the sterile inner container 20 with a minimal risk of contamination.

The first tray 15 features a peripheral edge section. In addition, a bead for grasping is formed at both end sections 11 and 12, which borders on the edge section. An inner surface at the first end section 11 is inclined at a shallow angle of about ten (10) to thirty (30) degrees, preferably approx. fifteen (15) to twenty-five (25) degrees, to define the tilted position of the inner container 20 relative to the outer container 10.

FIG. 4 shows a perspective view of the interior container 20 with a tilted carrier 30 retaining a sterile bone screw 40. To hold the bone screw 40, the carrier 30 features a molded part 35 in which the bone screw 40 is clamped to prevent the bone screw 40 from falling out.

The carrier 30 can be accommodated in a second receiving compartment 23 of the second tray 25. In FIG. 4, the carrier 30 is tilted relative to the inner container 20 by applying pressure to the first end section 22. For this purpose, the carrier 30 can feature a beveled surface at a corresponding first end section, which rests on an inner surface of the receiving compartment 23 in the tilted state. The beveled surface thereby defines a tilt angle for the carrier 30 relative to the inner container 20.

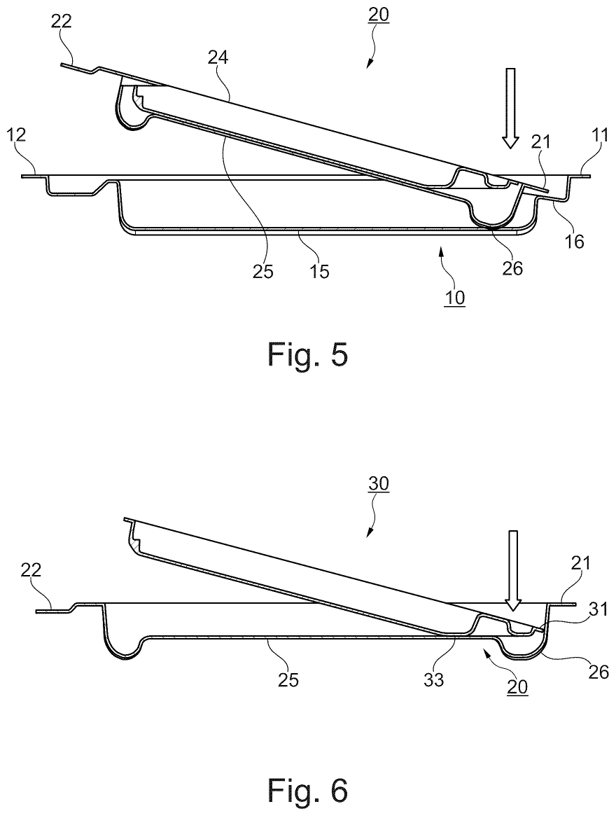

FIG. 5 shows a sectional view through the outer container 10 with the inner container 20 tilted. FIG. 6 shows a sectional view through the inner container 20 with the carrier 30 tilted.

In FIG. 5, the indentation 26 of the second recess can be seen, which rests as a pivotal point for the tilting movement of the inner container 20 relative to the outer container 10 on the surface of the first recess in the first receiving compartment 13. By pressing (see arrow in FIG. 5) on the first end section 21 of the inner container 20, the opposite second end section 22 tilts out of the outer container 10. The tilting movement takes place around the indentation 26, which rests on the bottom of the recess 15 of the outer container 10.

Furthermore, the outer container 10 features a beveled surface 16 on the first end section 11, which serves as a stop for the first end section 21 of the inner container 20 and defines an end position of the tilting movement, so that the tilted inner container 20 assumes a predetermined and stable tilted position, which facilitates the removal of the inner container 20 and prevents the inner container 20 from dropping down.

Tilting the carrier 30 out of the inner container 20 works in a similar manner. By pressing on the first end section 21 of the inner container 20 and on the first end section 31 of the carrier 30, the carrier performs a tilting movement around one end of a beveled surface 33 at the base of the carrier 30. The end of the beveled surface 33 rests on the base in the recess 25 of the inner container 20 and serves as a pivot for the tilting movement. The beveled surface 33, similar to the beveled surface 16 of the outer container 10, defines an end position of the tilting movement so that the carrier 30 can be held securely and stably in the tilted position.

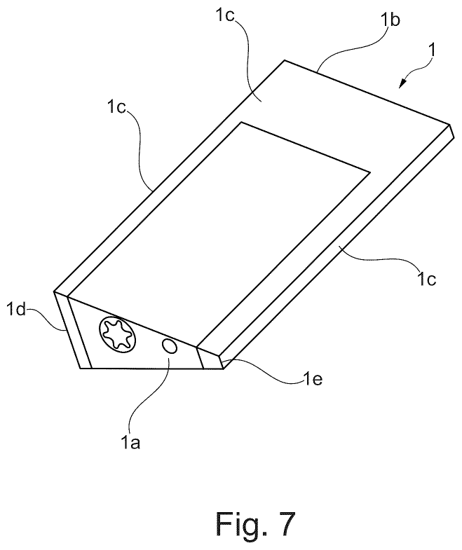

FIG. 7 shows a further example embodiment of the outer packaging 1 of the sterile packaging. In this example embodiment, the outer packaging features the shape of a prism, which features two square side faces 1a, 1b. The lateral face 1a forms the bottom face of the prism. The lateral face 1b forms the top face of the prism. The lateral surface area is formed by four rectangular and right-angled lateral faces 1c. Two opposing face lengths 1e, 1d of the bottom face 1a have different lengths.

The terms “left hand” and “right hand” are not meant to be restrictive, but are merely intended as examples. It is understood that the user's other hand can also perform the respective task in each case.

In the presented description, several features have been labeled with the terms “first” and “second”. These designations only serve to clearly distinguish the individual features. In particular, no spatial or functional arrangement or prioritization should be inferred from this.

If a list of alternatives is followed by “or” in this application, it should be understood that both the listed alternatives by themselves and, if appropriate, a combination of several or all listed alternatives are to be understood.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

1-10. canceled

11. A sterile packaging for a sterile object (40), comprising:
an outer packaging (1);
at least one container (10) arranged in the outer packaging (1), the least one container (10) forming a sterile barrier, the least one container (10) forming a receiving compartment (13) within the least one container (10), wherein the least one container (10) comprises a first tray (15) closed by a peel-off first lid (14) and includes a first recess within which the receiving compartment (13) is formedm
wherein the outer packaging (1) is formed as a prism with two triangular or rectangular lateral faces (1a, 1b) that are positioned opposite each other along a longitudinal direction of the outer packaging (1) and that form a bottom face (1a) and a top face (1b) of the prism and with three or four rectangular lateral faces (1c) forming a lateral surface area of the prism.

12. The sterile packaging of claim 11, wherein:
the bottom face (1a) and the top face (1b) are formed as a rectangular prism; and
two opposite lateral lengths (1d, 1e) of the bottom face (1a) and the top face (1b) have different lengths such that one of the lateral lengths (1d) is at least three times as long as that of the other lateral lengths (1e).

13. The sterile packaging of claim 11, wherein the bottom face (1a) and the top face (1b) are shaped as a triangle.

14. The sterile packaging of claim 13, wherein the bottom face (1a) and the top face (1b) are shaped as a right-angled triangle.

15. The sterile packaging of claim 11, wherein one or both of:
the outer packaging (1) comprises a predetermined breaking point line (2) for opening the outer packaging (1); and
the outer packaging (1) comprises a transparent window (3).

16. The sterile packaging of claim 11, wherein:
the at least one container (10) comprises an outer container forming a first sterile barrier and the receiving compartment (13) forms a first receiving compartment;
the at least one container (10) further comprises an inner container (20) arranged in the first receiving compartment (13), forms a second sterile barrier, and forms a second receiving compartment (23);
the outer container (10) comprises a first tray (15) closed by a peel-off first cover (14) with a first recess in which the first receiving compartment (13) is positioned;
the inner container (20) comprises a second tray (25) closed by a peel-off second cover (24) with a second recess in which the second receiving compartment (23) is positioned; and
the second recess comprises an indentation (26) that rests on a surface of the first recess in the first receiving compartment (13) and is configured as a pivotal point for a tilting movement of the inner container (20) relative to the outer container (10).

17. The sterile packaging of claim 16, wherein:
the first tray (15) comprises a peripheral edge section (17) that surrounds the first recess and to which the peel-off first cover (14) is attached; and
the second tray (25) comprises a peripheral edge section (27) that surrounds the second recess and to which the peel-off second cover (24) is attached.

18. The sterile packaging of claim 16, wherein:
the sterile packaging extends along the longitudinal direction;
the first tray (15) features a first end section (11) for holding the outer container (10) and a second end section (12) opposite the first end section (11) in the longitudinal direction;
the first recess is formed in the longitudinal direction between the first end section (11) and the second end section (12); and
a thinned section (90) is formed between the second end section (12) and the first recess to allow for creasing of the second end section (12).

19. The sterile packaging of claim 16, wherein a carrier (30) is arranged in the second receiving compartment (23) and comprises a shaped part (35) with a third receiving compartment for the sterile object (40).

20. The sterile packaging of claim 19, wherein one or both of:
the shaped part (35) exerts a clamping action on the sterile object (40); and
the sterile object (40) is a bone screw.
